(19) United States
(12) Patent Application Publication
(10) Pub. No.: US 2003/0191458 A1
(43) Pub. Date: Oct. 9, 2003

(54) Light-activated drug delivery method and device

(76) Inventors: Cornelius Diamond, San Diego, CA (US); Scott Arouh, San Diego, CA (US)

Correspondence Address: FUESS & DAVIDENAS, Suite II-G, 10951 Sorrento Valley Road, San Diego, CA, 92121-1613, US

(21) Appl. No.: 10/116,699

(22) Filed: Apr. 3, 2002

(51) Int. Cl. A61B 18/18

(52) U.S. Cl. 606/14; 607/2; 606/2; 604/20; 424/423

(57) ABSTRACT

A capsule, or a matrix, of a substance, most typically a polymer, that is degraded by a photo-acid or, less preferably, by a photo-base, physically contains or incorporates (i) a photo-acid, or a photo-base, or precursors to same, and (ii) one or more molecular agents, normally drugs. Placed in vivo, the photo-acid or photo-base or its precursors is (are) changed into an acid or base, as the case may be, by impinging radiation, most preferably by one or more light beams of green or longer wavelengths to which tissues are transparent, or else x-rays. The preferred light beams are two in number, spatially and temporally intersecting to produce the acid or base in vivo at precise regions and times by process of two-photon absorption. The photogenerated acid or base ruptures or dissolves the containment capsules or matrix, loosing the contained molecular agents (i) at precise subcutaneous tissue locations (ii) at precise rates (iii) over precise time intervals.

### BACKGROUND OF THE INVENTION

1. Field of the Invention

The present invention generally concerns the controlled molecular release of an agent, normally a drug, in vivo.

The present invention particularly concerns methods and devices for photodynamically controlled release of molecular agents in vivo with a high degree of (i) spatial and (ii) temporal control.

2. Description of the Prior Art

2.1 General Methods, State of the Art, and Existing Validation(s), of the Regional Delivery of Pharmaceuticals

As reported in the article “Targeting tumors: More precise therapies aim to spare the body's healthy tissue” by Rachel K. Sobel appearing in U.S. News and World Report for Oct. 2, 2000, the traditional cancer therapies of chemotherapy and radiation have led to problems. They are thankfully potent, but at times too potent. Not only do they annihilate cancer cells; they wipe out healthy ones, too. This leads to devastating side effects.

For decades, researchers have pondered ways to soften these repercussions without diluting the treatments' strength. Drug dosage schedules have been varied to improve tolerance. New drugs have been designed drugs to counter nausea. But now, scientists have begun to take a vastly different approach to the toxicity problem: to aim treatments directly at tumors and spare healthy tissue along the way. In dozens of hospitals across the country, doctors are vigorously experimenting with novel “vehicles” such as glass beads and magnetic particles to deliver treatments right to the cancer.

The present invention will be seen to concern one such delivery vehicle, and the method of its use.

After discussing advances in radiation therapy including the “glass bead” containment of radioactive particles first approved by the FDA in 2000, Ms. Sobel further discusses advances in targeted chemotherapy, and particularly in chemical magnetism. While some doctors are trying to steer radiation to its target, others are striving to do the same with chemotherapy, reports Sobel. UCLA's Scott Goodwin has a gadget in mind. He is shuttling chemotherapy directly to tumors “with miniature magnetic vehicles—tiny medicine-coated particles made of iron dipped in doxonibicin, a strong anti-cancer agent. Once injected into the bloodstream, these particles gravitate toward the tumor by the pull of a magnet, the size of a large soup can, positioned over the tumor site for 15 minutes. “The particles are actually pulled through the blood vessel wall out into the tumor itself,” says Goodwill, principal investigator of the trial. “Then you get a slow release of the drug inside the tumor tissue.”

Whether this technique will prove superior to traditional treatments is yet to be determined. So far, preliminary results with liver cancer suggest it might beneficially be used in the lungs, pancreas, and brain. The treatment minimizes side effects such as hair loss and nausea. And it leaves a stockpile of toxic drugs in the tumor for a few days with minimal effect on rest of the body. This is a welcome change from standard chemotherapy, where only a fraction of the drug dose congregates at the desired tumor site; the rest circulates in the blood stream and unnecessarily harms healthy tissues. So, there are legitimate reasons to think the magnetic transporters might be more effective, says Goodwin, but “that's a long ways from saying that we know it's better.”

The present invention will be seen not to require the involvement of any other method, nor the apparatus of any other method, to target the delivery of pharmaceuticals in vivo. However, the present invention will also seen not to conflict with the simultaneous, or sequential, use of another delivery method, and/or another apparatus—including magnetic particles and magnetic delivery methods. Combination of previous methods and apparatus with the method and apparatus of the present invention will be seen to be synergistic. For example, magnetism can be used to draw a drug delivery vehicle of the present invention—soon to be disclosed—to a target site (therein to exist in higher, or much higher, concentration than other locations within the animal) while the release mechanism of the present invention—soon to be disclosed—can then be applied to release the drug from the delivery vehicle with complete control as to the time(s) and amount(s) of release(s).

The chemical targeting of anti-cancer chemotherapy is also reported by Ms. Sobel in the context of an Ohio woman who thought she had squelched her breast cancer with radiation, chemotherapy, and mastectomy only to have a plum-size lump soon surface. Her doctor injected the tumor locally with an experimental viscous gel because the tumor had only popped up in one place. Treating the whole body would have been like “killing something with a sledge-hammer when, in the scheme of things, it's the size of a grain of rice,” says Richard Leavitt, an oncologist and vice president for gel-producer Matrix Pharmaceutical of Fremont, Calif. The tumor shrank in a matter of weeks, thanks to the gel's clever cocktail of ingredients. The gel contains chemotherapy blended with epinephrine, which constricts blood vessels surrounding the tumors. The taut blood vessels trap the chemotherapy at the site for hours, or even days. As a result, drug concentration can be 10 to 1,000 times higher than with bloodstream administration.

Again, the chemical targeting of tumors will be seen not to be necessary for use of the present invention: but the present invention is fully compatible with the chemical targeting of tumors and can, indeed, deliver to the chemicals to the tumor site with enhanced precision and selectivity.

More than 450 patients have been treated so far for melanoma, breast, colon, liver, and esophageal cancer, among others. And for head and neck cancer patients, nearly 1 out of 3 saw tumor size shrink by at least half, “Many of these patients had failed standard chemotherapy, but this actually worked,” says Glenn Mills, professor of medicine at Louisiana State University Health Sciences Center at Shreveport.

Several other targeting methods have already been approved by the FDA, but doctors are still trying to Figure out how to effectively use them. Some scientists, for instance, are experimenting with cryosurgery, which entails freezing cancerous tissue to destroy it. Others are investigating radiofrequency (RF) ablation—poking tumors with probes to sear the cancerous tissue away with intense heat. “We know RF is safe in the liver, but we want to look at other organs, like the prostate gland, kidneys, and lungs,” says Steven Curley, a professor of surgery at the University of Texas M. D. Anderson Cancer Center.

In general, the in-vivo targeting of pharmaceuticals (and molecular probes, and RF ablation agents, etc.) may be said to have proven efficacy as a general concept, but the “devil is in the details” of delivery, and dosage, and times of dosage. It will be found that the present invention goes a good ways towards “blowing away” a “fog of imprecision” in the in-vivo internal application of pharmaceuticals, and to make this application equally as precise as, by way of example, the application of medicants to the surface of the body. Clearly the application of, for example, a salve to the skin is sharply precisely controllable in the area(s), time(s) and amount(s) of the application(s). So also will the present invention be seen to permit application of drugs and molecular agents inside the body with equal precision as they are applied on the surface of the body.

2.2 The Efficacy of the Regional In-vivo Delivery of Pharmaceuticals

Significant advances have been made in the discovery and development of novel drugs; however, selective delivery of these drugs to specific therapeutic sites in the body has not been improved (circa 2000) beyond the approaches discussed in the previous section 2.1. Many drugs are limited in their effectiveness due to delivery of (i) sub-optimal drug concentrations coupled with (ii) short persistence durations at the desired site. In addition, the permissible dosages of freely-circulating drugs are often limited due to severe systemic side effects.

Light-induced in vivo drug delivery offers the potential to specifically target the action of pharmaceuticals to occur regionally anywhere within the body, and only at the time or times that specific target areas are illuminated with light.

In another area, the localization of the process of drug delivery is invaluable to the drug discovery process. This process consists of three essential and expensive steps: target discovery, lead identification, and clinical testing. The latter two steps render unusable many potential drug candidates. If the application of drugs could be localized and, to a lessor extent, precisely controlled as to amount—as are both taught by the present invention—then portions of these steps could be skipped, weeding our unsuitable drug candidates more quickly and increasing the proportion of viable drug candidates.

2.3 Photodynamic Therapy

To this end of photodynamically controlling (or at least moderating) the delivery of drugs, linear optical excitation of molecular agents has been extensively studied.

See, for example, Kennedy et al. (J. C, Kennedy, R. Potter and D. C. Ross, “Photodynamic Therapy with Endogenous Protoporphyrin IX: Basic Principles and Present Clinical Experience,” Journal of Photochemistry and Photobiology, B: Biology, 6 (1990) 143-48). Kennedy, et al., give an overview on development and application of several photosensitive molecular agents for clinical treatment of disease. However, the performance of these agents and methods have met with limited success.

See further, for example, A. R. Young, “Photocarcinogeniety of Psoralens Used in PUVA Treatment: Present Status in Mouse and Man,” Journal of Photochemistry and Photobiology, B; Biology, 6 (1990) pp. 237-247. Young presents studies strongly suggesting that the optical radiation used in common treatment regimes using linear optical excitation of photosensitive molecular agents can itself produce disease and detrimental side effects. The method of the present invention will be found to employ optical radiation to which the tissues of the body are substantially transparent, and which is therefore noninjurious, or at least minimally injurious.

Nonlinear methods of exciting chemical reactions have been employed to improve spatial selectivity and to overcome the limitations inherent in linear phototherapy. See, for example, M. J. Wirth and R. E. Lytle, “Two-Photon Excited Molecular Fluorescence in Optically Dense Media,” Analytical Chemistry 49, (1977) pp. 2054-2057. Wirth and Lytle teach the use of two-photon optical excitation as a means for stimulating target molecules present in optically dense media. The present invention will also be seen to permit use of two-photon absorption during the photodynamically-controlled release of drugs, although not in the manner taught by Wirth and Lytle.

See also W. D. Pfeffer and E. S. Yeung, “Laser Two-Photon Excited Fluorescence Detector for Microbore Liquid Chromatography,” Analytical Chemistry, 58 (1986) pp. 2103-2105. Pfeffer and Yeung discuss the advantages of using two-photon excitation of molecular agents to aid in the selectivity of the analysis in highly scattering, low target populated environments. These dual advantages of two-photon absorption—selectivity, and suitability even for sparse targets—will be seen to be used to good effect in the method of the present invention.

2.4 Encapsulation of Pharmaceuticals

The present invention will be seen to involve a very particular form of encapsulation.

The encapsulation of pharmaceuticals, and the methods of encapsulation, are well known, with much research focused on the creation of micro structures or vesicles derived from natural and synthetic materials. These vesicles are generally made of materials having a high amphophilic lipid content, for example, surfactants or phospholipids. Lipid microcylinders self-assemble spontaneously from diacetylenic phospholipids under appropriate physicochemical conditions. The microcylinders are typically 50-500 microns in length and 0.5-1 micron in diameter, with walls that consist of helically wrapped layers of lipid. The microcylinders can be freeze-dried and reconstituted with the bioagent of choice. The microcylinders can also be reconstituted with polymer solutions to create microcylinder composites with modified release rates.

These lipid structures generally fall into two classes: (1) muhilamellar vesicles, which are composed of a series of spherical shells formed of lipid bilayers interspersed with aqueous layers and ranging in diameter from 0.1-4 μm, (2) unilamellar vesicles, which have a lipid bilayer surrounding a large, unstructured aqueous phase and range in diameter from less than 0.2 μm to greater than 1 μm. Multilamellar vesicles are used for sustained release of reactive materials, while unilamellar vesicles are desirable for the encapsulation of large molecules such as enzymes.

The major roadblock in preventing widespread use of such encapsulation techniques is that they are relatively unstable toward mechanical, chemical and physical stresses. This, combined with the lack of an effective method of in vivo release, has made lipid encapsulation unsuitable for use in vivo (circa 2000).

Methods for improved stability have included the incorporation of proteins, sugars and cholesterol as is taught in U.S. Pat. No. 4,900,566 to Wheatley, et al. This patent additionally discloses—among other methods such as ion concentration and temperature perturbations—a method to initiate lipid disruption by 360 nm light.

Likewise, U.S. Pat. No. 5,366,881 to Singh and Schnur shows the incorporation of polymer in the creation of such vesicles in an effort to enhance stability. In particular, this patent discloses a method of using the cis to trans isomerization of adiazobenzene-incorporated lipid to open channels in bilayers of the lipid-polymer vesicles in the presence of UV radiation. However, animal tissue is highly absorbent to UV radiation, and to use such methods of release in vivo would be highly destructive to such tissue. In addition, it is desirable to produce large concentrations of a pharmaceutical agent at a localized spot on a rapid timescale; the lipid-encapsulation method fails to produce such concentrations because diffusion through a lipid layer of the encapsulated material occurs on too slow a timescale for appreciable benefit.

Another, alternate, method relevant to the drug delivery method of the present invention has attempted to address these issues. Magnetic dissolution of carbon particulates which have a pharmaceutical agent absorbed onto their surface is addressed by W. W. Schutt, et al., in “Biocompatible magnetic polymer carriers for in vivo radionuclide delivery,” Artificial Organs, 23, 98-103, January 1999. While the method of Schutt, et al. does allow site-specific targeting by a magnetic field exterior to the body, placement of the particulates is by a catheter and is limited in localization by the size of the magnetic field. The field is typically on the order of 10 centimeters in diameter, so spreading the released drugs over a targeted area much less than this size could potentially destroy otherwise healthy tissue.

Still another method relevant to the drug delivery method of the present invention is that described in U.S. Pat. No. 5,829,448 to Fisher, et al. This patent describes a technique in which there is multi-photon activation of a molecular agent in vivo. In particular, a photoactive molecular agent is dispersed throughout the tissue, wherein the desired region to be treated, which may be at a significant depth below the surface of the tissue, retains a portion of this photoactive molecular agent. This region is irradiated with light of a wavelength sufficient to penetrate the surface of the tissue, typically near infrared radiation (NIR). At sufficiently high intensity, this light excites a two-photon transition in the photoactive molecular agent, causing the agent to initiate its designated function. This technique of Fisher, et al. has the advantage of localization of the reaction to the region where the intensity is above the two-photon transition level. The drawbacks of such a method are that (i) the photoactive agent has to be stable to chemical, thermal and mechanical stresses so as not to be destroyed before activation, and (ii) it must be concentrated in the desired region of treatment in sufficient quantities for efficacy. In addition, it limits treatment to molecular agents that have a photoactive component, which eliminates drug candidates on the basis of uptake in the body and toxic effects rather efficacy on the target. Still further, such photoactive compounds are often difficult to design.

While the substantial body of prior art exemplified by these cited examples give many options for radiation-induced drug delivery, a method for combining the most attractive features of various drug delivery techniques has not previously been taught. Specifically, combining (i) the spatial selectivity of nonlinear excitation with (ii) the ability to disseminate in vivo molecular agents which are not photo-active, has not previously been taught.

Therefore, it is an object of the present invention to provide a method for the in vivo dissemination of molecular agents including drugs in plant or animal tissue with a high degree of spatial, and temporal, selectivity. This selectivity means that the molecular agents, or drugs, are not released where not desired to be released, or when not desired to be released, but are regularly and reliably released exactly where, and exactly when, desired to be so released—even in vivo at some depth within bodily tissues.

The molecular agents, or drugs, need not be, and are not, themselves photoactive. The molecular agents, or drugs, need not be, and are not, necessary of being combined with, or in the presence of, anything photoactive in order to perform their function. The molecular agents or drugs are simply that: agents or drugs used for therapy, probes or other biological purposes where there is absolutely no intent or requirement that these agents or drugs should incorporate still other properties especially including phototropic or photochromic properties. These other properties are in any case beyond present knowledge to incorporate (circa 2000), and highly likely of being forever impossible, or nearly impossible, of incorporation if the function of the agent or drug is to be left substantially intact. In simplest terms, it is neither sensible, prudent nor easy to alter molecular agents or drugs having biological effect(s)—lest these effects be themselves undesirably altered—and the present invention eschews that they should be in any way altered.

It is a further object of the present invention to provide a method using one or more light sources each of a wavelength that is less likely to be absorbed or scattered by plant or animal tissue than are the wavelengths of light used in present methods.

It is yet another object of the present invention to provide a general method for (i) the encapsulation of a molecular agent or drug, and (ii) its subsequent release from encapsulation in vivo.

It is still another object of the present invention to provide a method for allowing release of multiple molecular agents from encapsulation within an a single individual capsule, allowing for multiple therapeutic functionalities from the same device. It is an object of the present invention that different functionalities may be activated in concert, or in series, as desired.

Consideration of this specification, including the several Figures and examples to follow, will enable one skilled in the art to determine additional objects and advantages of the invention,

### SUMMARY OF THE INVENTION

The present invention contemplates the precise molecular delivery of one or more agents of biological effect, normally drugs, in vivo by isolating the agents through physical mechanical encapsulation and/or containment within a matrix until, at one or more highly spatially selectable sites at one or more selectable times, the one or more agents are loosed from their isolation by radiative, or by photo, processes—particularly including non-linear photo-excitation processes.

The present invention is based on the observation that it is a lot easier to design as photochemical system suitable for use in vivo, and for containment of biologically reactive agents, if the chemistry of the photochemical system need not itself link the chemistry of the agent or agents, which can be any of innumerable drugs undergoing innumerable chemical reactions. Indeed, in the photochemical drug delivery system of the present invention the photochemical components preferably do not chemically react, link or compound the biological agents, but are rather inert to these agents, and serve only to controllably physically, and only rarely chemically, isolate and contain the agents either by encapsulation of by holding the agents in(side) or within a matrix. When the encapsulation is photochemically penetrated, or the structure of the matrix impaired—at a level that may not, and may desirably not, rise to obliteration but only functional corruption—then the contained agent or agents is (are) loosed.

The unique features of the present invention offer distinct advantages for the treatment of disease in humans and animals. First, precise amounts of agents—which agents do not themselves have to have photoactive properties, and which most commonly do not have photoactive properties—may be delivered to desired areas and volumes of an organism. The agents are loosed from their encapsulation or containment by illumination of their encapsulating capsule or matrix, both preferably in the form of a microsphere, with radiation, preferably light radiation. In accordance with the present invention, the radiation preferably impairs, or even obliterates, the capsule or containment by producing a photo-acid. The produced photoacid acts to reliably, and most normally controllably progressively, degrade or destroy the containment function of the capsule or matrix and therein loose the agent, with which agent the photo-acid does not interfere.

Furthermore, and preferably, the regional radiation, or photo, illumination is non-linear, and is preferably realized with two intersecting light beans that induce two-photon absorption in the target photo-acid (which is within the shell of the capsule, or which forms the body of the matrix). Design of a practical and effective two-photon is not particularly easy, requiring as it does one of the relatively rare substances having a relatively large two-photon absorption cross section in order to work. (Two-photon absorption is controlled by the well-know quantum mechanical equations of multi-photon absorption, and is about 10-11 less effective than is one photon absorption.) The point is not that two-photon systems are either unwieldy difficult, or ineffective—which they are not—but rather that, should this requirement also being added to a preferred photochemical binder within a light-activated drug delivery system, then it would be almost impossible to expect the binder to also exhibit suitable chemistry with the drug. Of course, in the present invention the binder need not exhibit any chemistry with the drug, which it only serves to contain and to which it is substantially inert. Thus the concept of the present invention that the photochemical system is desirably chemically unreactive with the drug, which it only serves to selectively contain, is reinforced.

But why the preference for non-linear, two-photon, absorption? The preferred two-photon process provides several advantages. Most importantly, each of illuminating light beams is of a wavelength—relatively longer in accordance with the principles of two photon absorption, and relatively less energetic in accordance with the laws of physics—that will most typically pass easily through, and be completely non-interactive with, the tissues of the body. The illuminating light beams—although typically both visible and intense as best facilitates alignment—normally do not appreciably interact with anything until and unless they become spatially and temporally intersecting, and then only to particular molecules—such as those of the preferred photoacid—that present a high cross-section to two-photon absorption.

The dual illuminating light beams also provide for precise spatial and temporal control of the generally progressive (i) breaching of the micro-capsules or (ii) break-up of the matrix, and the loosing of the molecular agents or drugs. The two-photon process particularly permits that common molecular agents and drugs may be precisely released from their encapsulation or matrix containment in precise subcutaneous tissue locations at precise rates over precise time intervals.

Accordingly, the entire process of the in-vivo placement of agents in accordance with the present invention is very precise—regardless of precisely what the one or more delivered agents of biological effect are. The areal and volumetric discrimination in placement is very great: virtually all of the agents are initially released where and when desired, and virtually no agents are released anywhere else nor at any other time. Indeed, micro-capsules that are not intentionally breached by photo-reaction of photo-acid precursor molecules within the shells of the micro-capsules or material of the containment matrix are normally, typically and desirably excreted from the body, making that contents of excreted unbroken micro-capsules or un-deteriorated matrices are never even released in the body.

The particular locales and volumes where the encapsulated agents or drugs are released is very precisely controllable: there is no difficulty in placement of the agents with spatial tolerances on the order of millimeters. The amount of the agents or drugs released is very precisely controllable, and the release of the agents may be halted and resumed indefinitely many times, over time. Reference is typically made to graphs and tables derived from experimentation on laboratory animals in order to determine the amount (s) of agent release attendant upon different intensities and durations of illumination at different sites within the organism. Thus the rate, as well as the times, of the release of the one or more agents is highly controllable. The agents may be released, for example, in a “pulsating” pattern so that agent density peaks, and peaks locally, when and where the agents are released, with a minimal accumulation and/or minimally-sustained concentration of the agents within the entire organism.

Clearly the present invention supports the process of placing a bolus of a highly toxic agent at the site of, for example, a cancer, after which time natural in vivo dispersion will occur. If it is of any use, the toxic agent can sometimes be made to be reactive with another chemical (not of the photochemical system) for purposes of neutralization or enhanced elimination from the body. In such case the volume ringing, or spherically enclosing, the treatment area may be impregnated with this “take-up” agent, including again by the method of the present invention! Thus as the toxic agent delivered “on target” by the method of the present invention ultimately dissipates, it can itself be accurately, and timely, scavenged. More generally, by sequestering a drug at a target site, the localized concentration remains high while circulating levels are minimized in the body.

Perhaps the best way to think about the control accorded by the present invention is to compare it to that control that is exercised when a physician treats a surface wound on the body. When the wound is on the hand then the physician does not apply salve to the foot. When the physician applies the salve, he normally does not apply it to appreciably more area than the area of the wound. (Indeed, it may be useful to keep surrounding tissues unaffected by the salve so that they may more effectively “grow into” the area of the wound.) Even in the area of the wound, the physician does not apply the salve at indiscriminate thickness, but applies it is such amount as is calculated to be most efficacious. When a single topical application of salve is ill-calculated to protect the wound over a prolonged period of healing, the physician will likely call for periodic re-application of the salve. If the patient develops an allergy to the salve, the physician will cease to apply it, or if continued application of the salve outweighs the detriment, to apply the salve at times and amounts best calculated to minimized adverse side effects (such as when the patient is sleeping). All this precision, which is taken for granted as “common sense” in the treatment of surface wounds, has not heretofore been possible in the treatment of internal conditions.

The present invention clearly starts to accord the same precision in the treatment of the interior of an organism as has previously routinely been applied in treatment of the surface of the organism. When, where, and how much of an agent of biological effect is applied is all precisely controllable.

Furthermore, note was made in the Background of the Invention section of this application that the ability to target a pharmaceutical on the bodily organs and afflictions with which the pharmaceutical purports to deal has definite advantages in the drug discovery process. To continue the example, it is not desirable to clinically investigate the effect of a particular salve on an abrasion wound of the hand only by applying the salve to a fungus on the patient's foot.

1. A Method of Delivering a Drug to a Specific Therapeutic Site in an Animal—Materials Aspects

Therefore, in one of its aspects the present invention, is embodied in a method of delivering an agent of biological effect, typically a drug, to a specific therapeutic site in an animal. Stressing the aspects of the various materials that permit this method to be performed, the preferred method is as follows.

The preferred method involves mechanically physically isolating a drug within a vehicle (i) stable to contain the drug in the animal en route to and at a therapeutic site, (ii) but susceptible of having its containment integrity corrupted by a chemical.

In previous drug delivery systems the chemical that corrupts the integrity of the containment could be delivered to the site by, for example, injection. However, in the present invention the therapeutic site is illuminated with radiation subsequent to the arrival of the drug within its containment vehicle so as to produce the chemical in vivo at the site. This production is done from photosensitive precursors of the chemical present at the site. The photo-produced chemical corrupts the containment and looses the drug.

The drug isolation and containment vehicle is normally either (i) encapsulation, or (ii) a matrix physically taking up and containing the drug. Both (i) encapsulation and (ii) matrix containment vehicles are preferably within a microsphere capable of traveling within a circulatory system of the animal. Encapsulation is preferably realized by coating the drug with a sheath coating. Matrix containment is preferably realized by treating porous polystyrene microspheres with acid until they swell and the drug is taken, it is theorized, into interstitial spaces from which it cannot thereafter escape until the polystyrene matrix is again swelled or else dissolved. Both the preferred (i) sheath coating, and (ii) matrix, are susceptible of being dissolved by an acid. The illuminating of the therapeutic site with radiation produces this acid.

Notably, the photosensitive acid precursors could be delivered to the site by numerous means, such as by injection. However, and quite logically, it is preferred to deliver the acid precursors by which, upon photoconversion to acid, the containment integrity of the (i) encapsulating sheath, or (ii) matrix, will be breached right within the (i) sheath or (ii) matrix itself. In this, preferred, manner of delivery the encapsulating, or the binding in a matrix, is more preferably with a sheath, or matrix, material also containing the precursor of the very chemical that is subsequently used to dissolve the sheath or matrix. Namely, the encapsulating or binding is preferably with a combination, stable in the animal en route to and at the therapeutic site, of (i) a substance that is dissolvable by an acid and (ii) a photo-acid sufficient to dissolve the substance when photo-activated but not otherwise. The illuminating of the therapeutic site thus photo-activates the photo-acid, permitting it to dissolve the substance and loose the drug.

The encapsulating or binding is still more preferably with a combination including a substance, dissolvable by the photo-acid, which consists essentially of a polymer. The illuminating of the therapeutic site thus photo-activates the photo-acid, permitting it to dissolve the polymer and loose the drug.

The illuminating of this photo-acid is preferably by process of two-photon absorption, which absorption is sufficient to activate the photo-acid, dissolve the polymer, and loose the drug.

More generally, the drug may be contained in or bound within or inside a matrix of any substance that is dissolvable by an acid. The encapsulation preferably contains both this substance and a photo-acid generator precursor of an acid that is sufficient to dissolve the substance. The encapsulation may in particular be plastic, preferably polystyrene and most preferably in the form of a microsphere. The photoacid precursor may in particular be a precursor to onium salt or to phosphoric acid. The illuminating of the therapeutic site then photo-activates the generator precursor, generating an acid that dissolves the substance and looses the drug.

The encapsulating or binding may be with a combination including (i) an ionic, or a (ii) non-ionic photo-acid generator precursor.

Conversely, the encapsulating or binding may be with a combination—stable in the animal en route to and at the therapeutic site—of (i) a substance dissolvable by a base and (ii) a photo-activated generator precursor of a base sufficient to dissolve the substance. In this case the illuminating of the therapeutic site activates the photo-activated generator precursor, generating a base that dissolves the substance and looses the drug.

As before (with the photo-generation of an acid) this illuminating of the photo-activated generator precursor is preferably by two-photon absorption sufficient to generate the base, dissolve the polymer and loose the drug.

In any (preferred) usage of two-photon absorption, the illuminating is preferably within the ultraviolet through green regions of the spectrum; radiation is which regions is poorly absorbed by animal soft tissue.

As far as materials go, the encapsulating is preferably within a polymer shell and the binding within a polymer matrix, more preferably a polymer shell or matrix drawn from the group consisting essentially of polystyrene and latex.

2. A Method of Delivering a Drug to a Specific Therapeutic Site in an Animal—Illumination Aspects

In another of its aspects the present invention is still embodied in a method of delivering a drug to a specific therapeutic site in an animal. Now stressing the aspects of the illumination that permits this method to be performed, the preferred method is as follows.

The preferred method is directed to delivering a chemical agent to a specifically selected therapeutic site within an animal. The method commences with, and by, holding the chemical agent in association with a shell or, equivalently, a matrix that is made from a polymer and a photosensitive precursor of an acid. This shell/matrix is stable to contain the chemical agent en route to and at the therapeutic site within the animal, but will lose integrity to hold the chemical agent when exposed to an acid form of the precursor.

The method continues by selectively illuminating the selected therapeutic site with two beams of radiation so as to produce by process of two-photon absorption acid from the acid precursor that is within shell/matrix present at the therapeutic site. The produced acid causes the shell/matrix to loose integrity to hold the chemical agent, loosing the chemical agent into the animal at the selected therapeutic site.

The chemical agent is physically mechanically held within (i) the shell, which may be hollow, or (ii) the matrix, which has interstitial spaces, by dint of being commingled with the polymer and the photosensitive acid precursor material of the shell/matrix. The shell/matrix thus looses integrity to hold the commingled chemical agent because it (the shell or matrix) may be said to “lose containment integrity”. The shell/matrix may, or may not, “dissolve”, the important thing being only that it loses capacity to contain the drug.

Note that the chemical agent and/or the photosensitive acid precursor, either of both, may be within the matrix by dint of being taken up within the (preferred) polymer material or the matrix, or encapsulated by the polymer material which serves as a shell. If the photoacid precursor is interior to a polymer shell, then this polymer must be in particular transparent to the thickness of the shell to the radiation(s) sufficient to activate the photoacid precursor. The shell may thus be eroded from the “inside out”, as well as in gross. In fact, there are many physical configurations by which a body may loose integrity to hold and contain a chemical agent. Regardless of the configuration employed, and including both matrix and encapsulating shell configuration, the breach of integrity by the photoacid—preferably produced by process of two photon-absorption—is most commonly total, effectively destroying the body and rendering incapable of containing a scintilla of the chemical agent.

The loosed chemical agent is most commonly a drug.

3. A Vehicle for Delivery of a Drug to a Therapeutic Site within an Animal

In yet another of its aspects the present invention may be considered to be embodied in a vehicle for delivery of a drug to a therapeutic site within an animal.

The vehicle preferably includes a drug-encapsulating sheath or matrix stable to contain a drug in the animal en route to and at the therapeutic site, but dissolvable by a chemical. This sheath preferably consists essentially of both (i) a substance stable to contain the drug in the animal en route to and at the therapeutic site but dissolvable by a chemical, and (ii) a photochemical, also stable in the animal en route to and at the therapeutic site, that is responsive to radiation for producing the chemical which will dissolve the substance, loosing the contained drug.

The (i) substance of the sheath or matrix more preferably consists essentially of a polymer, and still more preferably a polymer drawn from the group consisting essentially of polystyrene and latex.

The preferred polymer sheath or matrix is preferably dissolvable by an acid: the photochemical produces this acid. The photochemical is thus a photoacid, or a photo-acid generator.

The polymer sheath or matrix may alternatively be dissolvable by a base acid: the photochemical produces this base. The photochemical is thus a photo-base generator.

The radiation is preferably light radiation, including infrared light, but may also be x-ray and other radiation. Radiative energies sufficient to dissolve the substance are preferably produced by, and only by, process of two photon absorption as arises from the temporal and spatial coincidence of two radiation (normally laser light) beams. In accordance with the principles of two photon absorption, a single (laser light) radiation beam is preferably insufficient to dissolve the substance.

4. Methods Using X-rays Both With and Without a Chemical Reaction

In still yet another of its aspects the present invention may be considered to be embodied in a method of delivering a drug to a specific therapeutic site in an animal where the ultimate loosing of the drug is effected by x-ray radiation.

In the method a drug, substantially impervious to x-ray radiation, is contained within a body that is (i) stable to contain the drug in the animal en route to and at the therapeutic site but (ii) sensitive to x-ray radiation to substantially lose containment capability. The therapeutic site is illuminated with x-ray radiation so as to substantially destroy the containment capability of the body, loosing the drug.

The physically containing may be within a body also containing (i) a material more preferentially absorptive of x-ray radiation than is either the drug or tissues of the animal, and (ii) a agent sensitive to heat at a temperature higher than is the temperature of the animal to cause the body to substantially lose its containment capability. In this embodiment the illuminating of the therapeutic site with x-ray radiation heats the material until it emits such heat as does cause the agent that is sensitive to heat to substantially destroy the containment capability of the body, loosing the drug.

This physically containing within the body of the (i) material more preferentially absorptive of x-ray radiation than is either the drug or tissues of the animal is most preferably a containing of gold.

In one variant embodiment the illuminating of the therapeutic site with x-ray radiation will serve to heat the material—preferably gold—until it emits such heat as does directly cause breaking of chemical bonds of the body, loosing the drug.

In another variant embodiment the illuminating of the therapeutic site with x-ray radiation heats the material—preferably gold—until it emits such heat as does promote a chemical reaction, the chemical reaction serving to break chemical bonds of the body, loosing the drug.

5. Uses of The Present Invention

The methods, and delivery vehicles, of the present invention are suitable for the delivery of all types of small-and large molecule drugs, such as used in (but are not limited to) therapeutic applications in gene therapy, and infectious and vascular disease. In addition, the encapsulation of the present invention binds small molecules (immune suppressants, antibiotics anti-fungals, anti-cancer drugs), biologies (peptides), and genetic vectors (plasmids, virus, and anti-sense).

With regard to its advantages, the present invention generally provides for the treatment of a localized volume of animal or plant tissue by sequestering a drug at a desired site, permitting localized concentrations to be high while circulating levels are minimized in the body.

By providing a targeted drug delivery system, the present invention generally permits an lower overall dosage of drugs, while still achieving an efficacious concentration at the desired site in the body. This provides an opportunity to limit side effects created from nonspecific systemic exposure both by reducing both the total amount of drug administered as well as by limiting circulation of the drug throughout the body.

These and other aspects and attributes of the present invention will become increasingly clear upon reference to the following drawings and accompanying specification.

### BRIEF DESCRIPTION OF THE DRAWINGS

Referring particularly to the drawings for the purpose of illustration only and not to limit the scope of the invention in any way, these illustrations follow:

FIG. 1, consisting of FIGS. 1a and 1b, is and energy level diagram diagrammatically illustrating an example of a two-photon optical excitation.

FIG. 2 shows two different methods of inducing a two-photon reaction in a medium.

FIG. 3, consisting of FIGS. 1a and 1b, is (i) a diagram of the photoacid Benzil-Di-Methyl-Ketal, and (ii) a graph of an exemplary one-photon absorption spectrum of Benzil-Di-Methyl-Ketal of concentrations 0.0%, 0.1%, 3% in propanol for the optical wavelength range of 190-790 nm.

FIG. 4 is a graph of an absorption spectrum for animal tissue from the UV to NIR spectral region.

FIG. 5 is a graph of a sample scattering spectrum for animal tissue from the UV to NIR spectral region.

FIG. 6 is a diagram generally illustrating absorption of optical and x-ray radiation in tissue.

FIG. 7 is a diagram illustrating the extent of radiation spread in for various optical and magnetic dissolution processes.

FIG. 8 is a chart illustrating the size range of current polymer encapsulation techniques.

FIG. 9 is a diagrammatic illustration of a first preferred embodiment of the invention.

FIG. 10 is a diagram illustrating the multifunctional aspect of the invention.

FIG. 11 is a diagram illustrating the method of usage of the invention.

FIG. 12 is an illustration in parts A and B of operation of a second preferred embodiment of the invention for use with x-ray radiation.

### DESCRIPTION OF THE PREFERRED EMBODIMENT

The following description is of the best mode presently contemplated for the carrying out of the invention. This description is made for the purpose of illustrating the general principles of the invention, and is not to be taken in a limiting sense. The scope of the invention is best determined by reference to the appended claims.

The invention described here utilizes the unique physical properties of encapsulated photo-activated acid, in particular when activated by a non-linear optical process, to effect improved spatial control over the release of molecular agents in the body. The invention further has the advantage of being a photoactive release system where said molecular agents do not have to be restricted to the photoactive kind. The method is shown additionally to have the further advantages of reduction of tissue damage along the optical path, due to use of less harmful optical and x-ray wavelengths that have much reduced scatter and adsorption in tissue.

A major advantage of the invention taught within this disclosure is the usage of an acid that is two-photon photo-activated—thus a two-photon photo-activated acid—to dissolve a polymer sheath, or matrix, which encapsulates or otherwise holds or binds the molecular agent of interest, while not damaging said agent. The two spatially and temporally intersecting light beams not only define the volume(s) and the time(s) of any release(s) with precision, they pass easily, and non-detrimentally, through the tissues of the body. Controllable release of pharmaceuticals or like agents may thus be effected by the present invention inside virtually any tissue save bone.

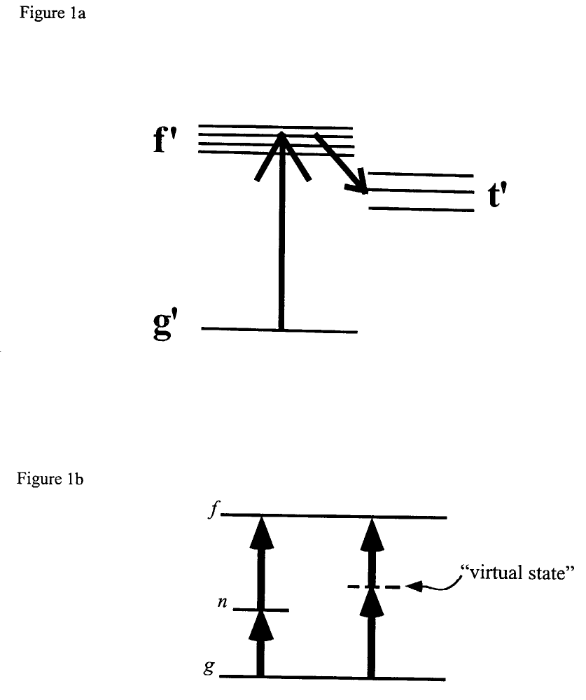

The salient features of the process of the present invention are more fully understandable by inspecting the following diagrams. FIG. 1 shows a simplified molecular energy diagram of a two-photon process, in where the vertical direction corresponds to a change in energy. The mathematical equation for cross-section of the two-photon process, as first written by Maria Geppart-Mayer in 1931 is

$${w}_{f ← g} = {ⅇ}^{4 \frac{{h}_{6}}{{λ}^{5}} {Γ}_{gf}^{- 1}} \times \vert {\sum }_{n} \frac{{( r \cdot {\hat{e}}_{1} )}_{gn} {( r \cdot {\hat{e}}_{2} )}_{nf}}{{ω}_{ng} - {ω}_{1}} + {\sum }_{n} \frac{{( r \cdot {\hat{e}}_{2} )}_{gn} {( r \cdot {\hat{e}}_{1} )}_{nf}}{{ω}_{ng} - {ω}_{2}} {\vert}^{2} \times \frac{1}{4} \vert {E}_{1} {\vert}^{2} \vert {E}_{2} {\vert}^{2}$$

A single photon can excite an electron from the first allowed electronic level g′ to an excited level if it has enough energy to do so. Level f′ is typically comprised of a band of allowed energy levels, divided and subdivided by their quantum-mechanically discrete vibrational and rotationally allowed states. Upon absorption the state can decay to, for example, a lower vibrational state t′, which typically happens on a timescale on the order of 10⁻¹²-10⁻¹⁵ seconds. The secondary state is usually stable on the order of 10⁻⁸ seconds.

This is called a linear excitation process, since this transition has a parity difference of 1 between the initial and final state. Level f can be excited by a two-photon process from level g in which the first photon is insufficient in energy for excitation to level f and only excites the photon to a virtual level n, which has a lifetime on the order of 10⁻¹⁵ seconds. If, in this time-period before de-excitation the level n is excited further by an additional photon of sufficient energy E₂≧E_{nf}, the energy required to excite the electron to the energy level f, the electron does not decay but is promoted to level f, promoted to a different level, photo-ionized or subject to further internal conversion and re-emission of energy.

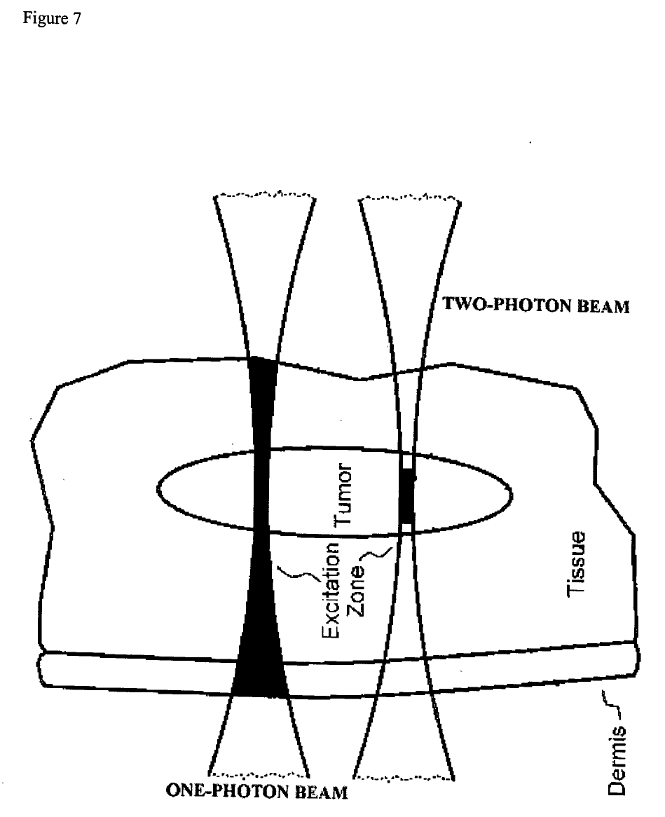

For the second process, the Fermi transition rate is given, and shown to be nonlinear in that it depends on the product of the number of photons I₁ and I₂ from the first and second energies. Because of this nonlinear dependence on intensity of the irradiating beam(s), it is possible that this threshold creates an interaction region which is smaller than the wavelength of light that is used, i.e. the interaction region is non-diffraction limited. Furthermore, unlike a linear intensity threshold, the interaction region is not spread along the path, as is illustrated in FIG. 7.

This leads to extremely fine spatial control as to the placement of the reaction so desired. Additionally, the two photons do not have to be of the same energies, and their respective polarizations are a factor in determining the strength of the interaction. Since a factor in the equation for the cross-section is comprised of the product of two dipole moments of the absorbing molecule, and this product is isomorphic to a quadrapole moment of the absorbing molecule, it is thus conclusive to look for molecules with large quadrapole moments when determining whether or not a molecule has a large two-photon absorption cross-section.

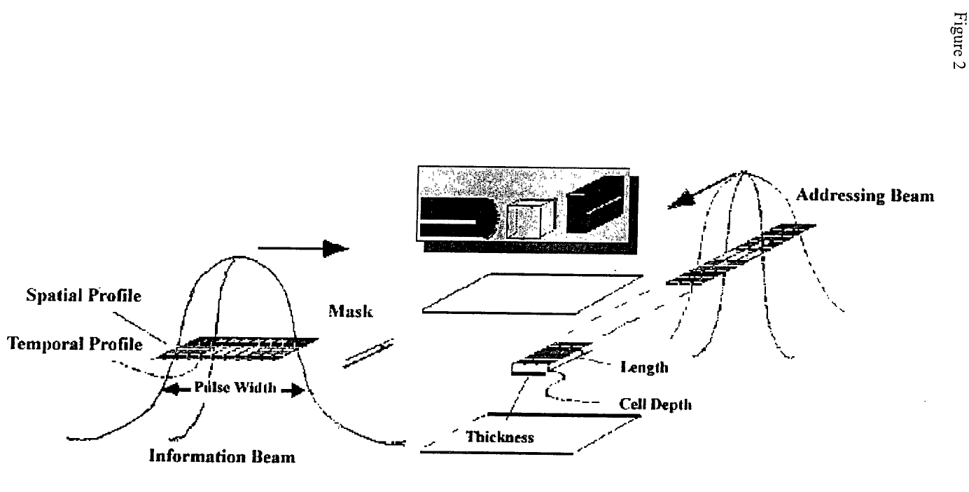

To target the desired region of tissue, there are two ways to enable intersection of two photons at the same spatial location. FIG. 2 shows these two ways: the first by the method of a single, tightly focused beam of sufficient intensity at the focal point and the second by the method of two intersecting beams, not necessarily of the same frequency. The first method has the advantage of sub-micron accuracy and easier alignment with to the targeted region, since there is only one beam to focus and the region of interaction is some fraction of the beam waist at focus. However, to achieve the high intensities necessary for two-photon interactions, the laser used for irradiation needs to be a pulsed laser with pulse widths that are of sub-picosecond duration. An example of this is a Titanium-Sapphire laser. In the second technique, the pulse duration can be several orders of magnitude higher, as the average power is usually much higher than with sub-picosecond pulsed sources. An example of a laser that produces such a pulse is a Nd:YAG laser. The beam is split and re-formed to intersect at the target volume. The advantage in this method is that one of the two beams can have a mask placed in front of the beam to tailor the interaction region, for example to simultaneously create two spatially disparate interaction regions. Since only the overlap of the two beams creates the interaction region, and the pulses used have more energy, the interaction region is usually larger than in the single beam case. In all cases, irrespective of method used, the two-photon activated acid determines the wavelength and energy needed for activation.

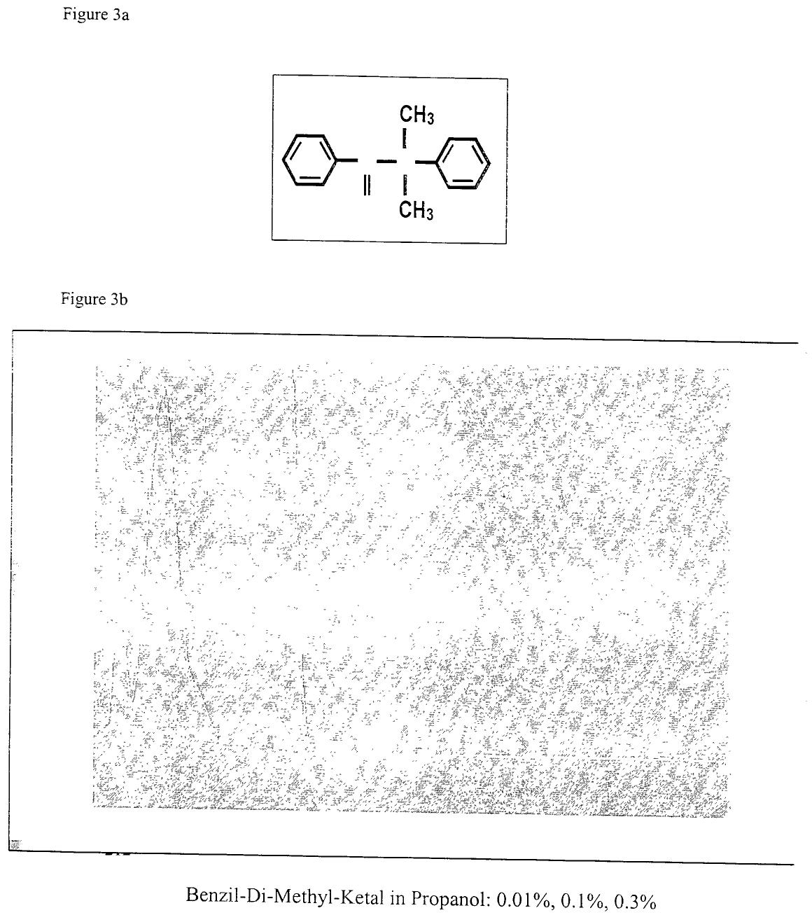

There are several types of photo-activated acids that can be used in the method described. FIG. 3 shows a typical one-photon absorption spectrum of the two-photon activated acid Benzil-Di-Methyl-Ketal in Propanol at concentrations of 0.01%, 0.1%, and 0.3% respectively, a larger absorption peak as the concentration is increased.

The chemical processes that occur in activating this acid are as follows.

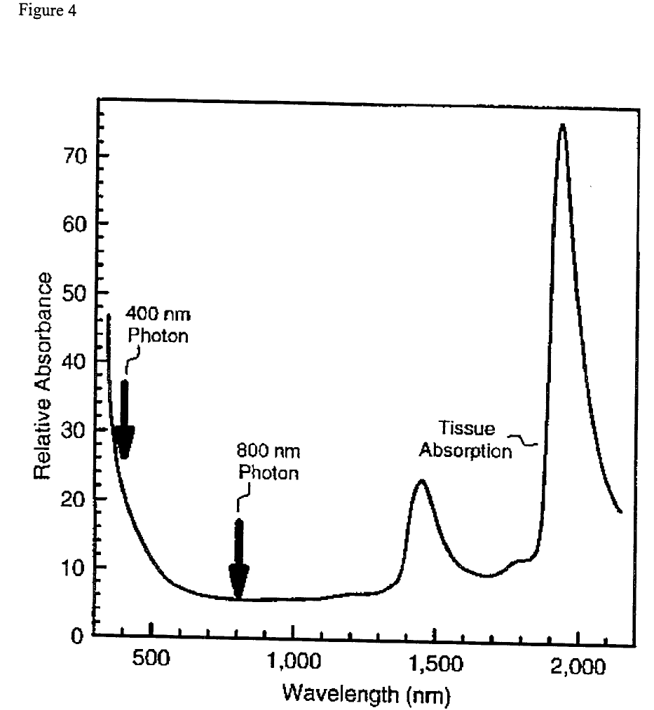

The activation of the photo-acid is usually in the green, blue or ultraviolet regions of the spectrum. However, it is seen in FIG. 4 that the absorption of tissue rules out the use of one-photon activation techniques in the ultraviolet (UV) parts of the spectrum for sub-tissue activation, since the majority of the light at, say, 400 nm is either absorbed or scattered in the outermost tissue layers. This absorption is due to excitation of molecules interior to surface tissue cells, such as pigments or proteins. This can lead to dangerous side effects such as tissue destruction and cancer.

In contrast, a near infrared (NIR) photon at 800 nm will have about a factor of four less absorption. Thus it is advantageous to use a two-photon process to excite a photochemical reaction over a one-photon excitation if less tissue damage is desirable. Finally, it is advantageous to use IR radiation as opposed to lower wavelengths in the visible or UV spectral regions since these later wavelengths can affect changes in the underlying molecular agent that is being released into the body. These molecular agents do not have large two-photon cross-sections, and thus are unaffected by IR or longer wavelengths of radiation.

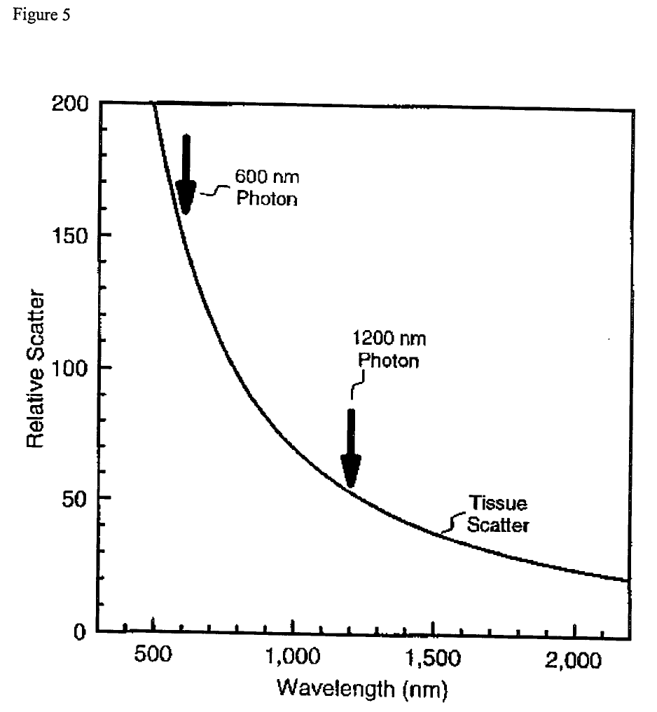

The advantage of using a longer wavelength two-photon excitation of a chemical reaction in tissue over that of a one-photon one is further seen in FIG. 5, which is a diagram of the normalized tissue scattering as a function of wavelength from the UV to the NIR part of the spectrum. It is seen even more strongly that scattering prevents use of the visible part of the spectrum for use of radiation to penetrate tissue.

Specifically, FIG. 5 shows that photons that correspond to typical excitation energies of photochemical reactions are highly scattered as compared to wavelengths at the lower end of the NIR and far infra-red (FIR) part of the spectrum. For instance, a photon of wavelength 1200 nm has a factor of about three less scatter in tissue than a photon of wavelength 600 nm. Thus while the cross-section for two-photon excitations is considerably reduced as compared to one-photon excitations, the absorption and scattering properties of tissue cause a two-photon excitation of a photo-activated agent, using IR radiation, to be favored, especially for sub-surface penetration.

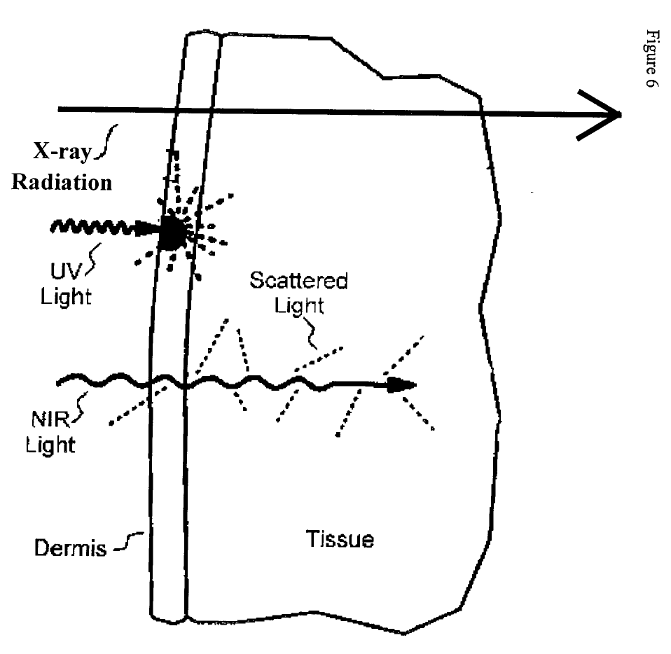

To further illustrate this point, FIG. 6 illustrates the penetration into tissue of visible, IR and X-ray radiation. It is seen that visible radiation is absorbed or scattered by the surface layers, while IR radiation penetrates the tissue layer to focus on the target sight. X-ray radiation does not focus at all and penetrates all tissue layers; only scattering off denser regions like bone. X-ray radiation can be collimated though by the placing of a vignette made of a highly scattering material such as lead in front of a diffuse beam of X-rays, as in the FIG. 6. Magnetic radiation cannot be collimated at all, and thus dissolution techniques of drug release based upon it cannot be localized to a region smaller than a few centimeters.

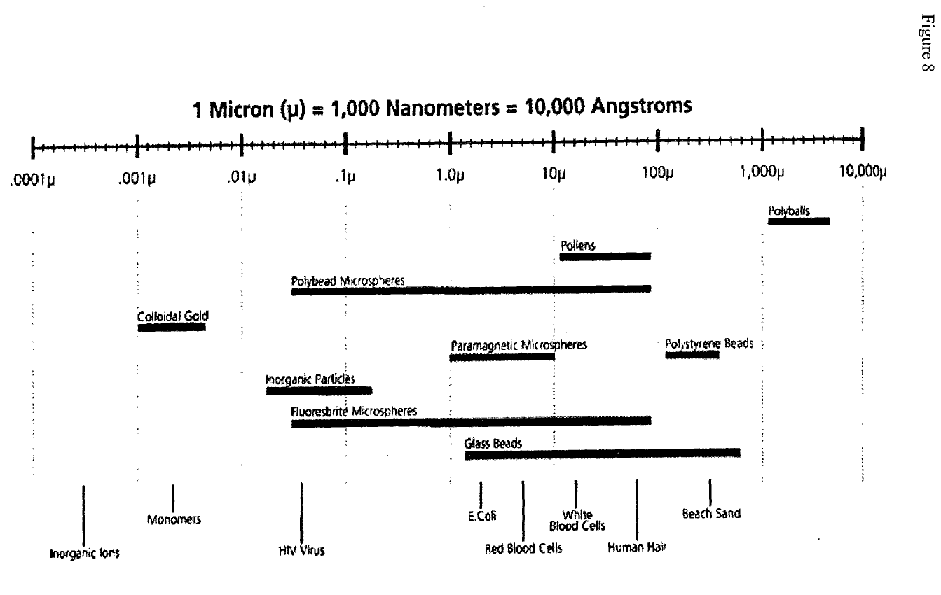

To be able to release all types of molecular agents, which come in a wide variety of sizes, it is useful to have a holder that can carry various sizes of such agents. FIG. 8 shows that polymer encapsulation can occur for objects over several orders of magnitude, ranging from polybead microspheres which can have diameters as small as 0.05 microns to microspheres, also known as polyballs, which can have diameters as large as 5 mm. For comparison, red and white blood cells are slightly smaller and larger than, respectively, 10 microns in diameter.

There are several instances where encapsulation of small particles is useful. For example, in gene therapy, it is important to deliver a genetically engineered virus to a target cell without that virus being destroyed by the body's natural defense mechanisms. Since most viruses are on the order of 50 nm in diameter, it may be seen that polymer encapsulation can readily be made small enough to incorporate single viral vectors in each microbead. This is important, as the encapsulated drug can be made small enough to penetrate cell walls, yet still contain the viral vector that will kill the cell upon release. Where and when it is desired to release large amounts of molecular agents in the general area of the targeted cells, the molecular agents themselves are engineered as to seek out and invest themselves in the correct cell, and polymer encapsulation techniques are able to create a vessel for large amounts of molecular agents as well.

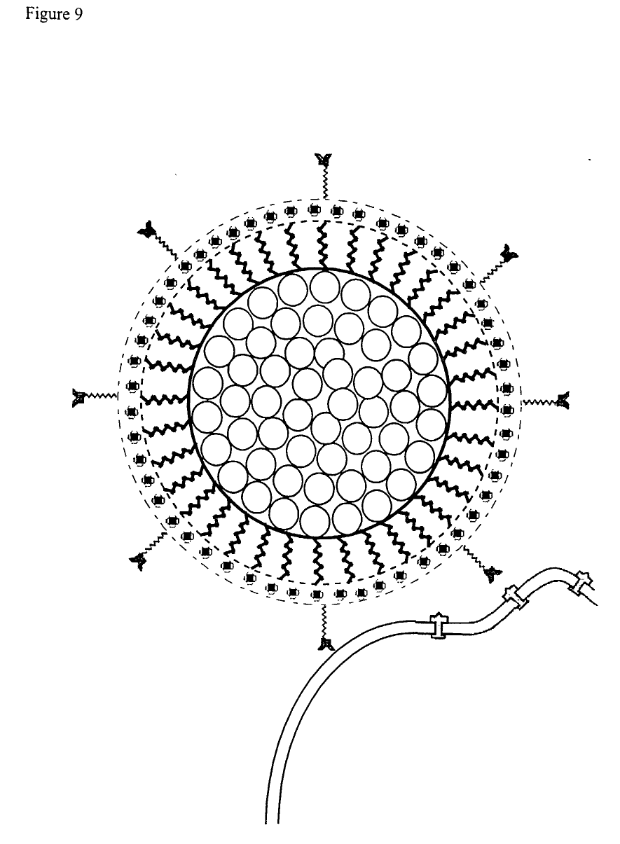

The light-activated encapsulated molecular agent delivery system is shown in FIG. 9. Numbering layers from the inside to the outside in a radial fashion, the first layer is an inner core of spheres that represent the molecular agent to be released in situ. The second layer surrounding the molecular agents is an encapsulating polymer coating that is biocompatible with plant and animal tissue. Further surrounding these two layers is a coating of two-photon activated acid. The acid precursors are either attached to the polymer coating or encapsulated again by a further surrounding layer of bid-compatible polymer shown as the fourth total layer counting outward. The layer is that of an optional targeting layer, attached to the underlying polymer, which would target attachment sites on the cell membrane. These targets would be tissue-specific, for example, so that molecular agents directed to liver tumors would not attached to gastrointestinal tissue. In the preferred embodiment, these attaching agents would be engineered into the molecular agent itself, but sometimes this is not possible due to the molecular functionality of the agent.

By layering in this manner, the tissue is only exposed during transit of the encapsulated molecular agent through said tissue to the biocompatible material, and is not exposed to the molecular agent itself until release.

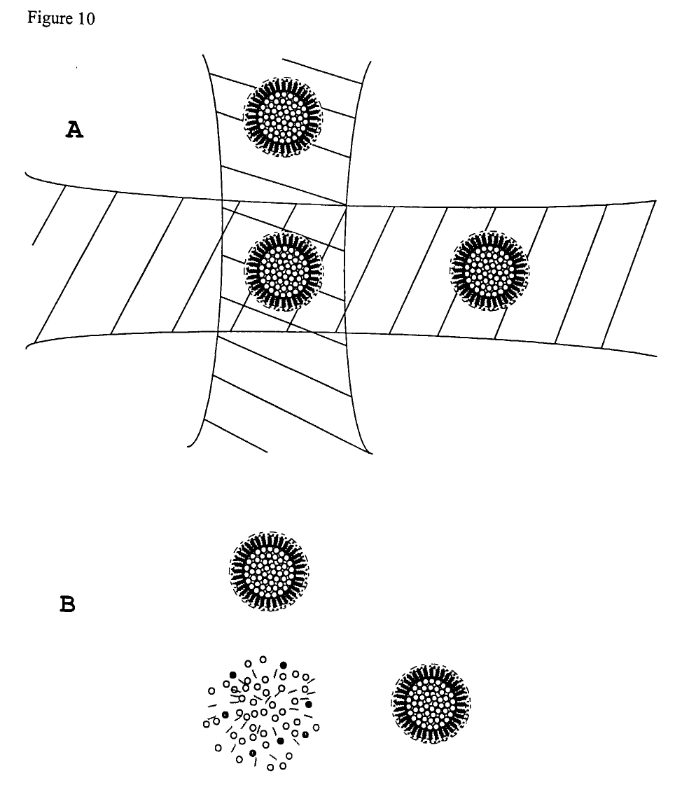

The method of the release is shown in FIG. 10. In part A, the encapsulated agent is stimulated by two overlapping laser beams. The intensity of each light beam is such that each is below the threshold for two-photon polymerization, but is so that the product of the two beams is greater than the two-photon polymerization.

In part B, the polymer shell of the encapsulated molecular agent that was in the convergence of the two light beams has been destroyed, releasing particles of the molecular agent, while the two encapsulated agents that were just illuminated by a single below-threshold laser beam are still intact. Thus the molecular agent is freed in a local area defined by the amount of diffusion at the site of beam overlap. Some of the molecular agents might be damaged by the acid layer or by the light as shown in part B, but their number will be in the minority as these effects cannot penetrate far into the core of the device, where the majority of the molecular agent is located.

In part B, part of the polymer and the acid precursor is still around, but as these are engineered to be biocompatible they have no effect on the toxicity of the local environment. The remaining molecular agents leave the body as waste and do not have any effect on the toxicity of the local environment. For certain molecular agents, there can be an additional deactivation step where in all areas of the body except the targeted area radiation can be applied to induce a chemical reaction in the molecular agent or destroying it outright, rendering excess amounts of the drug harmless to body tissue. Methods in which this can be carried out, for example, include the use of x-rays to break molecular bonds or the addition colloidal gold in the core of the light-activated drug release device which, upon stimulation from X-ray radiation, absorbs such radiation preferentially with respect to the surrounding environment. This radiation absorption causes the temperature of the gold molecules to increase to the point where the particles re-emit thermal radiation. This radiation raises the temperature of the molecular agent to the point where a pre-engineered chemical reaction occurs, de-activating the molecular agent either by bonding with another molecule or by the breaking of chemical bonds.

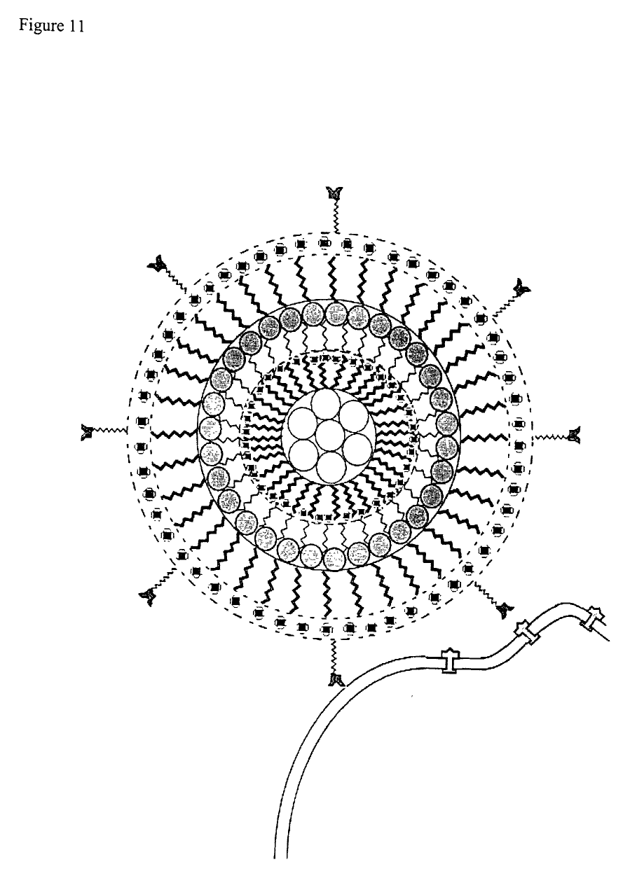

This method of layered encapsulation can be continued further, as shown in FIG. 11. The light activated drug delivery device has at its core the first molecular agent. This agent is surrounded by a polymer layer and an acid precursor layer as before. The acid precursor surrounding the first polymer layer has a two-photon cross-section at λ₁. This acid precursor is surrounded by another polymer layer and then a second molecular agent. This second molecular agent would be surrounded by a final polymer coating and a second acid precursor, however this acid precursor would have a two-photon sensitive to radiation at wavelength λ₂ which would be separated in the spectrum from light at λ₁ by at least the amount of distance such that the two-photon wavelength sensitivities of each acid precursor do not overlap.

The manner of the release would be as follows. The second acid precursor would be stimulated at wavelength λ₂ which would cause it to form an acid and dissolve the outer polymer sheath, releasing the second molecular agent. This agent would then diffuse in the local area of release. The use of such would be to pre-condition the tissue, for example, suppressing regulation of a certain gene expression in the local area of release.

The light-activated drug device would then be two-photon stimulated by radiation at wavelength λ₁. This would initiate the first acid precursor to become an acid and then dissolve the inner polymer sheath, releasing the first molecular agent. This first molecular agent would then diffuse in the local area to act upon the tissue that was preconditioned by the first agent, for example, to attach to a receptor site on the cell membrane which in concert with the stimulus provided by the first molecular agent kills the cell.

This process can be repeated with several more molecular agents, each with a photo-acid precursor-polymer-molecular agent-polymer surrounding shield. It is, however, anticipated that beyond a few layers it is easier just to administer another, separate light-activated molecular agent encapsulation device because of size and bioengineering concerns such as penetration into the bloodstream and number of photo-acid precursors which have unique two-photon activation wavelengths.

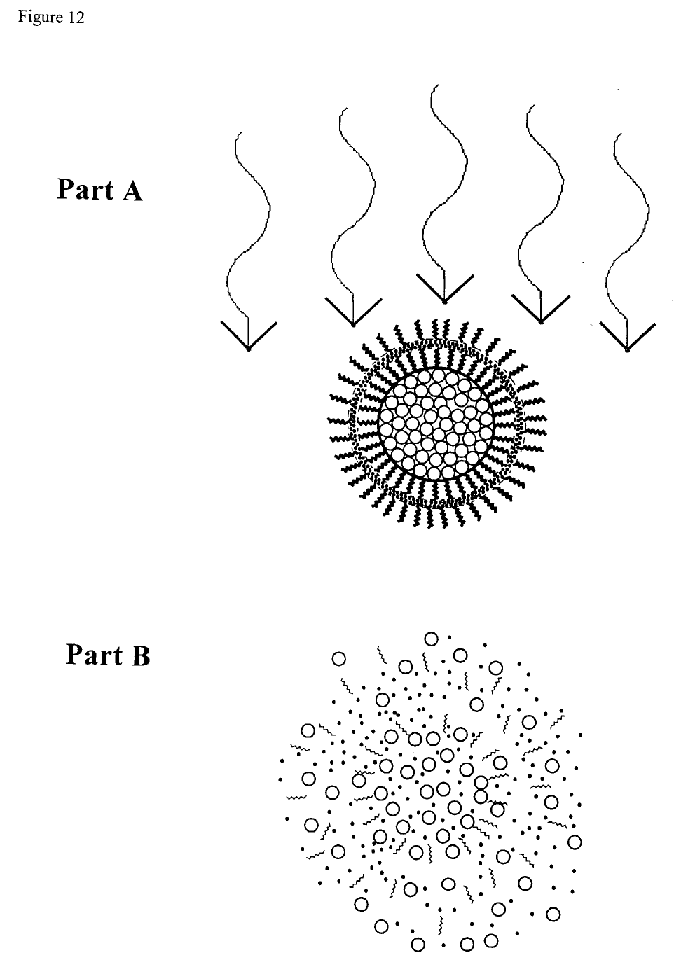

For distribution of molecular agents in tissue where there is (i) a need for large-area, rapid response, and/or tissue sensitivity to photoactive molecules, or for (ii) non-availability of a laser source at the correct wavelength for instituting a two-photon reaction, FIG. 12 shows the structure of the device to be used with X-ray radiation. The placement of the molecular agent is the same as before, at the core of the light activated drug delivery device. Surrounding this is a layer of biodegradable polymer. The next layer is colloidal gold particles, typically on the order of 10 nm in diameter, as shown in FIG. 8. Upon stimulation from X-ray radiation, the colloidal gold particles absorb such radiation preferentially with respect to the surrounding environment. This radiation absorption causes the temperature of the gold molecules to increase to the point where the particles re-emit thermal radiation that raises the temperature of the molecular agent to the point where an pre-engineered chemical reaction occurs, de-activating the molecular agent by bonding with another molecule or the breaking of chemical bonds.

This rise in temperature can also be used to initiate a chemical reaction in certain acid precursors, for use in situations where lower X-ray dosage is needed. In this manner the device is the same as in FIG. 10, however in the acid precursor layer colloidal gold particles are mixed in with said acid precursor. Upon irradiation the colloidal gold particles absorb X-rays and re-emit IR radiation. This emitted IR radiation is then absorbed by the acid precursor, stimulating the chemical reactions necessary for the active acid to form.

By this method, the temperature rise is localized to the light activated drug delivery device itself, and not to the surrounding local environment, allowing the device to carry out its functionality without destroying surrounding tissue (which would normally be destroyed by this rise in temperature).

2. Methods and Materials

In order to better facilitate the teaching of the present invention, the following are detailed description of the methods and materials used in the scope of the present invention.

2. Microsphere Technology

The method of encasing substances in micron-scale latex spheres is well developed. It is accessible to any person skilled in the art, and the methodology is given in U.S. Pat. No. 5,795/719, “Biotinylated Latex Microsphere, Process for the Preparation of Such a Microsphere and Use as Agent for Biological Detection” to J. Richard, et al. The Richard, et al., technology is briefly reviewed here.

A spherical shell is made of a polymer such as polystyrene or latex, in sizes as small as 50 nanometers. The polymers are made by polymerizing ethylenically unsaturated monomers, having functional groups at the surface. They are homopolymers or copolymers containing units derived from vinylaramatic monomers, ethylene monomers, ethylenic or alkanodic acids or esters, of which a proportion is functionalized. A few of them will now e mentioned, with no limitation being implied. They may be: (i) ethylene monomers of the isoprene, or acrylonitrile type; (ii) vinylaromatic monomers such as styrene, bromostyrene, chlorostyrene, vinyltoluene, or chloromethyl istyrene; (iii) alkemoic acids, esters or anhydrides such as acrylic or methacrylic acids, alkyl acrylates and methacrylates of which the alkyl group has 3 to 10 carbon atoms, hydroxyalkyl acrylates, acylamides, esters of ethylenic acids containing 4 or 5 carbon atoms; as well as (iv) difunctional monomers such as divinylbenzene and/or water-insoluble copolymerizable monomers.

A portion of the monomers carry groups capable of reacting, directly or indirectly, with functional groups—for example groups of the amine or carboxyl type—which are carried by biological molecules such as proteins and enzymes. Representatives of these functional groups, in which no limit is implied, include halogens, carboxyl, amine, aldchydic and sulfonyl groups, and epoxy and chloromethyl.

In this manner a given compound may also be chemically bound (covalently bonded) to the outer surface of a bead of either polymer or other materials such as glass or gold.

This technique is typically used to coat a sphere with an antigen that recognizes a specific protein. DNA, lectins (sugar-binding proteins), enzymes, and drugs of abuse have been similarly chemically bound to the surfaces of such particles. An extension of this technique consists of the use of a polymer containing magnetic particles, which permits the extraction of those proteins by application of a magnet field.

This technique is typically used to encase fluorescent markers for diagnostic assays of living biological specimens by several processes; one such being the method described in U.S. Pat. No. 5,462,866, “Semipermeable Microspheres Encapsulating Biological Material” to T. G. Wang. This Wang technique is not the preferred embodiment of the method of the present invention and is given for reference only.

4. Multi-photon Processes

Photon processes refer to the interaction of light (from a laser, for example) and a chemical substance (a photoinintiator). In the present invention photo processes are used to activate photo initiators, which in turn activate acid precursors and then acids. Appropriate photoinitiators and acid precursors are discussed in following sections in more detail; here we discuss the benefits of the use of multi-photon processes.

There are a variety of benefits from the use of multi-photon processes. First, one has greater spatial control of an activation area to more selectively deliver a therapeutic effect. This is because multi-photon activation limits the field of activation to the focus of the laser beam. This focus can be made exceedingly small, providing fine control over the location and localization of the resulting photodynamic effect.

Secondly, this localization in turn allows the delivery of large amounts of energy while minimizing damaging photo-thermal effects. The spatial resolution afforded allows smaller regions to be accessed than from one-photon techniques.

Thirdly, multi-photon processes also limit the amount of scatter of light that would otherwise damage tissue.

Fourthly, multi-photon processes allow deeper penetration. Multi-photon activation uses longer wavelength light that delivers activating energy more deeply within tissue than does normal (i.e., visible or UV) light.

Finally, multi-photon processes allow less tissue burning and more efficient use of radiation. Multi-photon processes allow the use of longer wavelengths than single-photon processes. Since the absorption spectrum of the body is significantly less at these longer wavelengths than at the frequencies typically used for single-photon processes, less of the energy goes into heating of the tissue for the multi-photon process, and more of the energy gets applied to the desired target process (of activating the photoactive acid precursor).

5. Photoinitiators

Photoinitiators are molecules thai gel excited by light stimulation. They may come in single- or multi-photon varieties. A single photon exciting an electron from a singled state to triplet state, which has a larger cross section for electron transfer with a colliding molecule, initiates single-photon photoinitiators. Examples of single-photon photoinitiators are Benzil and Rhodamine B.

Multi-photon photoinitiators operate in essentially the same way, except two or more photons are required to excite the electron from the singlet state to the triplet state. Examples of two-photon photoinitiators are the Ketal bis-donor derivatives discovered by Cumpston et al. See B. H. Cumpston, J, W. Perry, S. Marder et al, “New Photopolymers based on two-photon absorbing chromophores and application to three-dimensional microfabrication and optical storage”, Mat. Res. Soc. Symp. Proc, 488, 217-25, (1998). Also appropriate is Benzil-Di-Methyl-Ketal (BDMK) and the dye (6-benzothiazol-2-yl (2-naphthyl)) diphenylamine 8 (AF183). See Joshi, M. P., Pudavar, H. E, Swiatkiewicz, J, Prasad, P. N., and Reianhardt, B. A. “Three-dimensional optical circuitry using two-photon-assisted polymerization.” Applied Physics Letters, vol.74, (no.2), AIP, Jan. 11, 1999. pp. 170-2.

Photoinitiators are useful in that they allow chemical activity to be regulated by exposure of the molecules to light. This in turns allows a chemical process to be gated: upon exposure to light, the gate is open for this chemical process to proceed.

6. Photo-Acid and Photo-Base Generators

Compounds producing acids upon illumination with light are called photoacid generators, whereas those producing base—photobase generators. Compounds producing acids upon illumination with light are called photoacid generators (PAG), whereas those producing a base are called photobase generators (PBG).

Both PAG and PBG are suitable of application in the method of the present invention. PAGs are commonly used for polymer degradation, whereas PBGs are usually employed to inhibit acid diffusion.

There are two major groups of acid generators: ionic and non-ionic ones. One suitable group of ionic acid generators consists of onium salts containing metal halides (BF₄⁻, SbF₆⁻, AsF₆⁻ or PF₆⁻). Also suitable is aryldiazonium. See Spiess, W.; Lynch, T., Le Cornec, C., Escher, G.; Kinoshita, Y,; Kochan, J,; Kudo, T,; Masuda, S.; Mourier, T.; Nozaki, Y.; Olson, S,; Okazaki, H.; Padmanaban, M.; Pawlowski, G.; Przybilla, K. J.; Roschert, H-; Suehiro, N.; Vinet, F.; and Wengenrotb, H.: Evaluation results for the positive deep UV resist AZ DX 46, Proceedings of the SPIE (The International Society for Optical Engineering), vol. 2195. See also Advances in Resist Technology and Processing XI, San Jose, Calif., USA, Feb. 28-Mar. 1, 1994, 1994, p.84-95. Still further suitable are diaryliodonium, triarylsulfonium, and triaryl phosphonium salts. See S. P. Pappas, J-Imag. Tecnol 11, 146-157 (1985).

Upon irradiation with UV light of 190-300 nm wavelength the onium salts photoproduce a protic acid. See S. P. Pappas, op cit. Thermal stability is regarded as a major advantage of onium salts as photoacid generators.

Non-ionic photo-acid generators offer the routes of producing wide variety of acids, among which there are carboxylic, sulfonic, phosphoric acids as well as hydrogen halides. The lack of thermal stability is regarded as common disadvantage of non-ionic photoacid generators.

Incorporation of additives into polymer matrix can be accomplished by straightforward host-guest approach, as well as via chemical bonding of the photoactive compounds to the polymeric backbone [#8].

Many industrial applications of photoacid generators such as cationic polymerization, cross-linking of polymers, transformation of functional groups, and degradation of polymers exist. This last application—degradation of polymers—is directly related to the method of the present invention. See the next section 7. Polymer Degradation.

Efficient activation of the acid generator by two-photon, absorption is possible when excitation half-wavelength is close to the peak of the molecule's absorption and the two-photon cross-section is sufficiently large.

7. Polymer Degradation

Degradation of polymers by photogenerated acid occurs when either polymeric chain or polymer network crosslinks experience cleavage as a result of chemical reaction between active polymer groups and photogenerated protons. Such processes are widely used in positive photoresists technology. Due to specifics of in-vivo application under consideration, only systems working in the temperature range up to body temperature of 36.6° C. are of particular interest. Therefore systems where polymer degradation requires additional heating to temperatures up to 100-200° C. to trigger photoinduced acid catalyzed thermolysis are not suitable. See Ito, H.; Ueda, M.; Schwalm, R. Highly sensitive thermally developable positive resist systems. Journal of Vacuum Science & Technology B (Microelectronics Processing and Phenomena), vol.6, (no.6), (32nd International Symposium on Electron, Ion and Photon Beams, Fort Lauderdale, Fla., USA, May 31-Jun. 3, 1988.) November-December 1988, p. 2259-63. See also Ito H.; Ueda, M.; Renaldo, A. F. Thermally developable, positive tone, oxygen RIE barrier resist for bilayer lithography. Journal of the Electrochemical Society, vol.136, (no.1), January 1989, p. 245-249 (related is H.Ito and R. Shwalm, J. Electrochem. Soc. 136, 241-245 (1989)). See also Ito, H.; Ueda, M.; Schwalm, R. Highly sensitive thermally developable positive resist systems. Journal of Vacuum Science & Technology B (Microelectronics Processing and Phenomena), vol.6, (no.6), (32nd International Symposium on Electron, Ion and Photon Beams, Fort Lauderdale, Fla., USA, May 31-Jun. 3, 1988.) November-December 1988. p.2259-63. See also J. M. J. Frechet, M. Stanciulescu., T. Iizawa and C. G. Willson, Polym Mater. Sci. Engng 60, 170173 (1989). See also J. M. J. Frechet, B. Kryczka, S. Matuszczak, B. Reck, M. Stanciulescu and C. G. Willson, J. Photopolym. Sci. Technol. 3, 235-247 (1990). See also J. M. J. Frechet, J. Fahey, C. G. Willson, T. Iizawa, K. Igarashi and T. Nishikubo, Polym. Mater. Sci. Engng 60, 174-178 (1989). See also J. M. J. Frechet, C. G. Willson, T. Iizawa, T. Nishikubo, K. Igarashi and J. Fahey, Polymers in Microlithography Materials and Process; ACS Symposium Series 412 (E. Reichmanis, S. A. MacDonald, T. Iwayanagi Eds.), pp. 100-112, American Cernical Society, Washington, D.C. (1989). See finally Y. Inaki, M. Matsumura and K. Takemoto, Polymers for Microelectronics, ACS Symposium Series 537 (L. F. Thompson, C. G. Willson and S. Tagawa Eds.), pp.142-164, American Cemical Society, Washington, D.C. (1994).

Among those working at room temperature, examples of photodegradable systems are as follows.

Polycarbonates decompose to thymine photodimer and carbonate units upon irradiation in the presence of PAG. See H. Horito and Y. Inaki, J. Photopolym. Sci. I'echnol. 4, 33-40 (1991). #18. T. Nishikubo, T. Iizawa, Y. Sugawara and T. Shimokawa, J. Polym. Sci. Part A: Polym. Chem. 24, 1097-1108 (1986).

Photoinduced degradation of polymers containing vinyloxy groups in the presence of onium salts was demonstrated [#18]. Cleavage of crosslinked polymeric network by photogenerated acid is reported [#19]. See S. Moon, K. Naitoh and T. Yamaoka, Chem. Mater. 5, 1315 -1320 (1993).

Although specific embodiments of the invention will now be described with reference to the drawings, it should be understood that such embodiments are by way of example only and are merely illustrative of but a small number of the many possible specific embodiments to which the principles of the invention may be applied. Various changes and modifications obvious to one skilled in the art to which the invention pertains are deemed to be within the spirit, scope and contemplation of the invention as further defined in the appended claims.

8. Extensions of the Invention

In accordance with the preceding explanation, variations and adaptations of the light-activated drug delivery method and device in accordance with the present invention will suggest themselves to a practitioner of the medical arts. For example, an microsphere containing, or encapsulating, harmless iron as well as, preferably, a photoacid precursor, may be guided to a high concentration in a particular location by a magnetic field, with the (drug) contents of the microsphere being loosed by impinging radiation.

For example, two types of containment vehicles A, B with two types of photoacid precursors Aₚ, Bₚ, each precursor responsive to a particular energy radiation E_{A}, E_{B} may be simultaneously positioned in vivo, A one medical substance A_{S} that is within a one, A-type, containment vehicle is taken up, and/or rendered inert, and/or neutralized, and/or captured so that it may be efficiently excreted, by another chemical substance B_{S} that is within the second, B-type, containment vehicle. The A-type containment vehicle is first dissolved, normally by confluence of light radiation of energy E_{A} that is insufficiently energetic to cause the photoacid precursor A_{B} that is in or within the B-type containment vehicle to do, anything. Ergo the medical substance A_{S} that is within the A-type containment vehicle is loosed while the chemical substance B_{S} is still contained. Then radiation of energy E_{B} is applied. This does nothing to the photoacid precursor Aₚ, and the A-type containment vehicle that has not already been done. But it now activates the B_{P} photoacid precursor, ruptures or dissolves the B-type containment vehicle, and looses the chemical substance B_{S} to interact with the medical substance A_{S}. The interaction is normally so as to cease an interaction with the animal and the animal's tissues that is too extreme to be unduly prolonged, or for which further prolongation is irrelevant and deleterious.

In accordance with these and other possible variations and adaptations of the present invention, the scope of the invention should be determined in accordance with the following claims, only, and not solely in accordance with that embodiment within which the invention has been taught.

What is claimed is:

1. A method of delivering an agent of biological effect to a specific therapeutic site in an animal comprising:
physically containing within a body both (i) an agent of biological effect, and (ii) photosensitive precursors of a chemical suitable to destroy containment integrity of body, the body being stable to contain the agent stable in the animal en route to and at the therapeutic site while the agent is also stable in presence of the photosensitive precursors co-contained within the body;
illuminating the therapeutic site with radiation so as to produce from photosensitive precursors the chemical at the site of radiation, the chemical destroying containment integrity of the body and loosing the agent of biological effect.

2. The drug delivery method according to claim 1 wherein the physically containing within a body comprises:
encapsulating within a sheath (i) the agent of biological effect, and (ii) the photosensitive precursors, the sheath stable to maintain encapsulated both (i) the agent of biological effect and (ii) the photosensitive precursors in the animal en route to and at the therapeutic site but the sheath being susceptible of being dissolved by a chemical; and
wherein the illuminating the therapeutic site with radiation is so as to produce the chemical destroying containment of the encapsulating sheath by dint of perforating the sheath, permitting the loosing of the agent through, and at a rate determined by, perforation occurring to the sheath.

3. The drug delivery method according to claim 2 wherein the physically containing within a body comprises:
encapsulating within a sheath (i) the agent of biological effect, and (ii) the photosensitive precursors, the sheath stable to maintain encapsulated both (i) the agent, and (ii) the photosensitive precursors, in the animal en route to and at the therapeutic site but susceptible of being dissolved by a chemical; and
wherein the illuminating the therapeutic site with radiation is so as to produce the chemical destroying containment of the encapsulating sheath by dint of effectively substantially disintegrating the encapsulating sheath, effectively loosing all the agent by removing any containment of the agent.

4. The drug delivery method according to claim 1 wherein the physically containing within a body comprises:
physically binding within a matrix stable to contain (i) the agent of biological effect, and (ii) the photosensitive precursors, the matrix stable to maintain bound both (i) the agent and (ii) the photosensitive precursors in the animal en route to and at the therapeutic site but susceptible of being dissolved by a chemical; and
wherein the illuminating the therapeutic site with radiation is so as to produce the chemical eroding containment of the matrix by dint of loosing its physical binding of the agent, permitting the agent to escape containment at a rate determined by the loosing of the binding.

5. The drug delivery method according to claim 1 wherein the physically containing within a body comprises:
physically binding within a matrix stable to contain (i) the agent of biological effect, and (ii) the photosensitive precursors, the matrix stable to maintain bound both (i) the agent and (ii) the photosensitive precursors in the animal en route to and at the therapeutic site but susceptible of being dissolved by a chemical; and
wherein the illuminating the therapeutic site with radiation is so as to produce the chemical destroying containment of the matrix by dint of negating its binding of the agent, effectively loosing all the agent.

6. The drug delivery method according to claim 1 wherein the illuminating the therapeutic site with radiation produces a chemical that progressively erodes containment by the body, progressively loosing the agent of biological effect.

7. The drug delivery method according to claim 1 wherein the illuminating the therapeutic site with radiation produces a chemical destroying containment by the body, effectively immediately loosing all the agent of biological effect.

8. The drug delivery method according to claim 1
wherein the containing is within a microsphere body capable of traveling within a circulatory system of the animal.

9. The drug delivery method according to claim 1
wherein the containing is of (i) the agent of biological effect and (ii) photoactive precursors of an acid, within a body susceptible of being dissolved by the acid; and
wherein the illuminating of the therapeutic site with radiation is so as to produce from the precursors the acid that dissolves the body, and looses the agent.

10. The drug delivery method according to claim 9
wherein the containing is within an acid-dissolvable polymer body; and
wherein the illuminating of the therapeutic site with radiation is so as to produce from the precursors the acid dissolving the polymer body, and loosing the agent.

11. The drug delivery method according to claim 9
wherein the containing is with an ionic photoacid precursor.

12. The drug delivery method according to claim 9
wherein the containing is with a non-ionic photoacid precursor.

13. The drug delivery method according to claim 10
wherein the illuminating of the precursors is by two-photon absorption sufficient to (i) produce from the precursors the acid, (ii) dissolve the polymer, and (iii) loose the agent.

14. The drug delivery method according to claim 1
wherein the containing is of (i) the agent of biological effect and (ii) a photoacid, within a body susceptible of being dissolved by photo-activated acid; and
wherein the illuminating of the therapeutic site with radiation is so as to activate the photoacid, dissolving the body and loosing the agent.

15. The drug delivery method according to claim 1
wherein the containing is of (i) the agent of biological effect and (ii) photoactive precursors of a base, within a body susceptible of being dissolved by the base; and
wherein the illuminating of the therapeutic site with radiation is so as to produce from the precursors the base that dissolves the body and looses the agent.

16. The drug delivery method according to claim 15
wherein the containing is within a base-dissolvable polymer body; and
wherein the illuminating of the therapeutic site with radiation is so as to produce from the precursors the base dissolving the polymer body, and loosing the agent.

17. The drug delivery method according to claim 15
wherein the illuminating of the precursors is by two-photon absorption sufficient to produce from the precursors the base that dissolves the polymer and looses the agent.

18. The drug delivery method according to claim 1
wherein the containing is of (i) the agent of biological effect and (ii) a photobase, within a body susceptible of being dissolved by photo-activated base; and
wherein the illuminating of the therapeutic site with radiation is so as to activate the photobase, dissolving the body and loosing the agent.

19. The drug delivery method according to claim 18
wherein the containing is within an base-dissolvable polymer body; and
wherein the illuminating of the therapeutic site with radiation is so as to activate the photobase, dissolving the polymer body and loosing the agent.

20. The drug delivery method according to claim 1
wherein the illuminating is with light radiation within the ultraviolet through green regions of the spectrum, which light radiation is poorly absorbed by animal soft tissue.

21. The drug delivery method according to claim 1 wherein the illuminating is by two-photon absorption sufficient to effect loss of containment integrity by the body.

22. The drug delivery method according to claim 1
wherein the agent encapsulated is itself unreactive with the illuminating radiation.

23. The drug delivery method according to claim 1
wherein the containment is within a substantially spherical microsphere body.

24. The drug delivery method according to claim 23
wherein the containment is within a substantially spherical microsphere body of diameter less than 1000 nanometers.

25. The drug delivery method according to claim 1
wherein the containment within is within polymer.

26. The drug delivery method according to claim 25
wherein the containment is within polymer drawn from the group consisting essentially of polystyrene and latex.

27. The drug delivery method according to claim 1
wherein the containment within the body is of an agent of biological effect in the form of a drug.

28. A method of delivering an agent of biological effect to a specific therapeutic site within an animal comprising:
containing the agent of biological effect within a body made from a (i) substance suitable to contain the agent of biological effect but susceptible to a chemical to lose containment integrity, and (ii) a photosensitive precursor of the chemical, the substance of the body being stable to contain the agent within the animal both en route to and at the therapeutic site but always susceptible to the chemical to lose containment integrity; and
illuminating the therapeutic site with radiation sufficient so as to produce from the chemical precursor the very chemical that causes lost of containment integrity of the substance of the body, therein loosing the agent of biological effect from the body into the animal at the therapeutic site.

29. The drug delivery method according to claim 28
wherein the illuminating of the photosensitive precursor is by two-photon absorption sufficient to generate the chemical from the photosensitive precursor.

30. The drug delivery method according to claim 28
wherein the containing of the agent of biological effect is by physically binding the agent within a matrix.

31. The drug delivery method according to claim 28
wherein the containing of the agent of biological effect is by physically encapsulating the agent within a shell.

32. A method of delivering a chemical agent to a specifically selected therapeutic site within an animal comprising:
holding the chemical agent in association with a matrix that is made from a polymer and a photosensitive precursor of an acid, the matrix being stable to contain the chemical agent both en route to and at the therapeutic site within the animal, but the matrix losing integrity to contain the chemical agent should the polymer from which the matrix is made become exposed to an acid derived from the precursor; and
selectively illuminating the selected therapeutic site with two beams of radiation so as to produce (i) by process of two-photon absorption acid, (ii) from the acid precursor that is within matrix present at the therapeutic site, the acid, this produced acid causing the polymer from which the matrix is made to lose integrity to contain the chemical agent and, consequently to this lost of containment integrity, loosing the chemical agent into the animal at the selected therapeutic site.

33. The method according to claim 32
wherein the chemical agent is held in association with the matrix by dint of being, along with the photosensitive acid precursor, physically contained within the matrix; and
wherein in presence of the acid the matrix looses integrity to contain the chemical agent because the matrix dissolves.

34. The method according to claim 32
wherein the chemical agent is held in association with the matrix by dint of being, along with the photosensitive acid precursor, encapsulated by the matrix serving as a shell; and
wherein in presence of the acid the shell matrix looses integrity to contain the chemical agent because the shell matrix perforates.

35. The method according to claim 34
wherein in presence of the acid the encapsulating shell matrix is not merely perforated, but is effectively destroyed and rendered incapable of containing even a scintilla of the chemical agent.

36. The method according to claim 32
wherein the loosed chemical agent is a drug.

37. A method of delivering a agent of biological effect to a specific therapeutic site in an animal comprising:
physically containing an agent of biological effect, which agent is substantially impervious to x-ray radiation, within a body that is (i) stable to contain the agent in an animal both en route to and at a therapeutic site, while remaining (ii) sensitive to x-ray radiation to substantially lose containment capability; and
illuminating the therapeutic site with x-ray radiation so as to substantially destroy the containment capability of the body, loosing the agent of biological effect.

38. The delivery method according to claim 37
wherein the physically containing is within a body also containing (i) a material that is more preferentially absorptive of x-ray radiation than is either the agent of biological effect or tissues of the animal, and also (ii) a material sensitive to heat at a temperature higher than is a temperature of the animal to cause the body to substantially lose its containment capability; and
wherein illuminating of the therapeutic site with x-ray radiation heats the material until it emits such heat as does cause the material that is sensitive to heat to substantially destroy the containment capability of the body, loosing the agent of biological effect.

39. The delivery method according to claim 38
wherein the physically containing within the body of the (i) material more preferentially absorptive of x-ray radiation than is either the agent of biological effect or the tissues of the animal is a containing of gold.

40. The delivery method according to claim 38
wherein the illuminating of the therapeutic site with x-ray radiation serves to heat the absorptive material until it emits such heat as does directly cause breaking of chemical bonds of the body, loosing the agent of biological effect.

41. The delivery method according to claim 38
wherein the illuminating of the therapeutic site with x-ray radiation heats the material until it emits such heat as does promote a chemical reaction that does break chemical bonds of the body, loosing the drug.

42. A vehicle for delivery of a drug to a therapeutic site within an animal, the vehicle comprising:
a drug-encapsulating sheath stable to contain a drug in an animal both en route to and at the therapeutic site but dissolvable by a chemical, the sheath consisting essentially of a binder material (i) stable to contain the drug in the animal both en route to and at the therapeutic site but (ii) dissolvable by a chemical, and
a photochemical, also stable in the animal both en route to and at the therapeutic site, responsive to radiation for producing the chemical which will dissolve the binder material, loosing the contained drug.

43. The vehicle for delivery of a drug according to claim 42
wherein the binder material of the sheath consists essentially of a polymer.

44. The vehicle for delivery of a drug according to claim 43
wherein the polymer sheath is drawn from the group consisting essentially of polystyrene and latex.

45. The vehicle for delivery of a drug according to claim 43
wherein the polymer sheath is dissolvable by an acid; and
wherein the photochemical is responsive to radiation to produce the acid.

46. The vehicle for delivery of a drug according to claim 45 wherein the photochemical producing the acid consists essentially of:
a photoacid.

47. The vehicle for delivery of a drug according to claim 45 wherein the photochemical producing the acid consists essentially of:
a photo-acid generator.

48. The vehicle for delivery of a drug according to claim 43 wherein the polymer sheath is dissolvable by a base; and
wherein the photochemical produces the base.

49. The vehicle for delivery of a drug according to claim 48 wherein the photochemical producing the base consists essentially of a photo-base generator.
